(19) United States
(12) Patent Application Publication
(10) Pub. No.: US 2012/0088891 A1
(43) Pub. Date: Apr. 12, 2012

(54) PROCESS FOR THE PREPARATION OF UNSATURATED ACYLAMIDOALKYLPOLYHYDROXY ACID AMIDES

(75) Inventors: Harald Keller, Ludwigshafen (DE); Mario Emmeluth, Bensheim (DE); Tim Balensiefer, Mannheim (DE); Paola Uribe Arocha, Mannheim (DE); Francesca Aulenta, Mannheim (DE)

(73) Assignee: BASF SE, Ludwigshafen (DE)

(21) Appl. No.: 13/264,634

(22) PCT Filed: Mar. 30, 2010
(86) PCT No.: PCT/EP10/54208

§ 371 Date: Dec. 29, 2011

(30) Foreign Application Priority Data
Apr. 15, 2009 (EP) 09157968.0

(51) Int. Cl. C08F 22/38; C08F 22/40; C07C 223/02; C07D 207/444; C08B 37/00; C07C 209/68

(52) U.S. Cl. 526/262; 536/18.7; 564/487; 564/502; 548/548; 526/304

(57) ABSTRACT

The present invention relates to a process for the preparation of unsaturated acylamidoalkylpolyhydroxy acid amides by reacting the reaction product of polyhydroxy acid lactone and aliphatic diamine with the anhydride of a monounsaturated carboxylic acid, to the unsaturated acylamidoalkylpolyhydroxy acid amides and also to a process for the preparation of polymers of unsaturated acylamidoalkylpolyhydroxy acid amides.

The invention relates to a process for the preparation of unsaturated acylamidoalkylpolyhydroxy acid amides, to the unsaturated acylamidoalkylpolyhydroxy acid amides and to a process for the preparation of polymers of unsaturated acylamidoalkylpolyhydroxy acid amides.

A process for the preparation of 1-amino-2-D-gluconoylaminoethane is described in H. U. Geyer, Chem. Ber. 1964, 2271.

U.S. Pat. No. 2,084,626 describes a process for the preparation of monoallylamide of gluconic acid. For the preparation, the lactone of gluconic acid is converted with allylamine in ethanol into the gluconamide.

Analogously to this, the preparation of the monoallylamine of lactobionic acid and its copolymerization with acrylamide is described by M. Chiara, M. Cretich, S. Riva, M. Casali, Electrophoesis (2001), 22, 699-706. The solvents used here then had to be removed by means of complex distillation.

DE 1 048 574 teaches the reaction of gluconolactone with aminoalkyl vinyl ethers to give the corresponding amides.

The targeted chemical synthesis of unsaturated acylamidoalkylpolyhydroxy acid amides is difficult on account of the high functionality of the sugar radicals.

It was an object of the invention to develop a process for the preparation of unsaturated acylamidoalkylpolyhydroxy acid amides which at least partly avoids the above-described disadvantages of the prior art. The synthesis should in particular be selective with a good yield of desired unsaturated acylamidoalkylpolyhydroxy acid amides, i.e. be able to be carried out without the formation of polyamides or polyesters and thus without the formation of a plurality of free-radically polymerizable double bonds in a cost-effective manner. The bond of the unsaturated carboxylic acid and the polyhydroxy acid lactone should have high hydrolysis stability. In addition, the preparation process should have a good space-time yield.

Accordingly, a process for the preparation of unsaturated acylamidoalkylpolyhydroxy acid amides has been found in which the reaction product of polyhydroxy acid lactone and aliphatic diamine is reacted with the anhydride of a monounsaturated carboxylic acid.

Furthermore, novel unsaturated acylamidoalkylpolyhydroxy acid amides have been found, and also polymers comprising acylamidoalkylpolyhydroxy acid amide groups in copolymerized form.

Preference is given to a process in which one or more polyhydroxy acid lactones are reacted with one or more aliphatic diamines in aqueous medium and the reaction product, preferably without interim isolation, is reacted with the anhydride of a monounsaturated carboxylic acid.

Schematically, the preparation takes place in two steps: in the first step of the reaction of the polyhydroxy acid lactone with the aliphatic diamine to give the corresponding aminoalkylaldonamide and in the second step of the reaction of the aminoalkylaldonamide with the anhydride of a monounsaturated carboxylic acid to give the unsaturated acylamidoalkylpolyhydroxy acid amide according to the invention. If desired, an interim isolation may be advantageous. However, the two process steps are preferably carried out directly in succession, i.e. without interim isolation.

Unless stated otherwise, within the context of this application, C₁-C₈-alkyl is methyl, ethyl, n- or isopropyl, n-, sec- or tert-butyl, n- or tert-amyl, and also n-hexyl, n-heptyl and n-octyl, and also the mono- or poly-branched analogs thereof. C₂-C₁₀-alkylene is preferably ethylene, propylene or 1- or 2-butylene.

Polyhydroxy acid lactones are to be understood below as meaning lactones of saccharides from natural and synthetic sources oxidized only on the anomeric carbon. Polyhydroxy acid lactones of this type can also be referred to as lactones of aldonic acids. The polyhydroxy acid lactones can be used individually or in their mixtures.

The saccharides are oxidized only selectively at the anomeric center. Processes for the selective oxidation are generally known and are described, for example, in J. Lönnegren, I. J. Goldstein, Methods Enzymology, 242 (1994) 116. For example, the oxidation can be carried out with iodine in an alkaline medium or with copper(II) salts.

The saccharides used for the preparation of the polyhydroxy acid lactones are open-chain and cyclic mono- or oligosaccharides from a natural or synthetic source which carry an aldehyde group in their open-chain form. In particular, the saccharides are selected from mono- and oligosaccharides in optically pure form. They are also suitable as stereoisomer mixture.

Monosaccharides are selected from aldoses, in particular aldopentoses and preferably aldohexoses. Suitable monosaccharides are, for example, arabinose, ribose, xylose, mannose, galactose and in particular glucose. Since the monosaccharides are reacted in aqueous solution, they are present, on account of the mutarotation, both in a ring-shaped hemiacetal form and also, to a certain percentage, also in open-chain aldehyde form.

Oligosaccharides are understood as meaning compounds with 2 to 20 repeat units. Preferred oligosaccharides are selected from di-, tri-, tetra-, penta-, and hexa-, hepta-, octa-, nona- and decasaccharides, preferably saccharides having 2 to 9 repeat units. The linkage within the chains takes place 1,4-glycosidically and optionally 1,6-glycosidically.

Preferably, the saccharides used are compounds of the general formula (I),

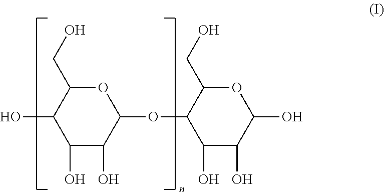

in which n is the number 0, 1, 2, 3, 4, 5, 6, 7 or 8. The resulting lactones here have the following formula (II)

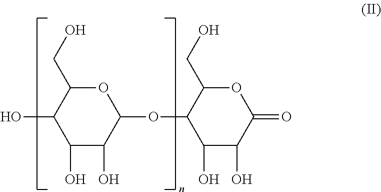

in which n is the number 0, 1, 2, 3, 4, 5, 6, 7 or 8.

The oligosaccharides in which n is an integer from 1 to 8 are particularly preferred. In this connection, it is possible to use oligosaccharides having a defined number of repeat units. Examples of oligosaccharides which may be mentioned are lactose, maltose, isomaltose, maltotriose, maltotetraose and maltopentaose.

Preferably, mixtures of oligosaccharides with a different number of repeat units are selected. Mixtures of this type are obtainable through hydrolysis of a polysaccharide, for example enzymatic hydrolysis of cellulose or starch or acid-catalyzed hydrolysis of cellulose or starch. Vegetable starch consists of amylose and amylopectin as main constituent of the starch. Amylose consists of predominantly unbranched chains of glucose molecules which are 1,4-glycosidically linked with one another. Amylopectin consists of branched chains in which, as well as the 1,4-glycosidic linkages, there are additionally 1,6-glycosidic linkages, which lead to branches. Also suitable according to the invention are hydrolysis products of amylopectin as starting compound for the process according to the invention and are encompassed by the definition of oligosaccharides.

Aliphatic diamines suitable according to the invention may be linear, cyclic or branched. Aliphatic diamines for the purposes of this invention are diamines with two primary or secondary amino groups, preferably with one primary and one further primary or secondary amino group, which are joined together by an aliphatic, preferably saturated, bivalent radical. The bivalent radical is generally an alkylene radical having preferably 2 to 10 carbon atoms which may be interrupted by O atoms and which can optionally carry one or two carboxyl groups, hydroxyl groups and/or carboxamide groups. Furthermore, aliphatic diamines are also understood as meaning cycloaliphatic diamines.

Aliphatic diamines substituted by hydroxyl, carboxyl or carboxamide that are suitable according to the invention are, for example, N-(2-aminoethyl)ethanolamine, 2,4-diaminobutyric acid or lysine.

The aliphatic diamines suitable according to the invention whose alkylene radical is interrupted by oxygen are preferably α, ω-polyetherdiamines in which the two amino groups are at the chain ends of the polyether. Polyetherdiamines are preferably the polyethers of ethylene oxide, of propylene oxide and of tetrahydrofuran. The molecular weights of the polyetherdiamines are in the range from 200-3000 g/mol, preferably in the range from 230-2000 g/mol.

Preference is given to using aliphatic C₂-C8-diamines and cycloaliphatic diamines, such as 1,2-diaminoethane, 1,3-diaminopropane, 1, 5-diaminopentane, 1,6-diaminohexane, N-methyl-1,3-diaminopropane, N-methyl-1,2-diaminoethane, 2,2-dimethylpropane-1,3-diamine, diaminocyclohexane, isophoronediamine and 4,4″-diaminodicyclohexyl-methane.

The anhydrides of a monounsaturated carboxylic acid used according to the invention are preferably selected from the anhydrides of C₁-C₆-alkyl-substituted acrylic acid, in particular acrylic anhydride, methacrylic anhydride, itaconic anhydride and maleic anhydride.

The reaction of polyhydroxy acid lactone with an aliphatic diamine generally takes place in an organic solvent or solvent mixture or in a mixture at least of one organic solvent with water. Suitable organic solvents are those which at 20° C. are miscible with water at least to a limited extent, in particular completely. This is understood as meaning a miscibility of at least 10% by volume of solvent, in particular at least 50% by volume of solvent in water at 20° C. By way of example, mention may be made of C₁-C3-alcohols, e.g. methanol, ethanol, propanol, isopropanol, ketones such as acetone, methyl ethyl ketone, mono-, oligo- or polyalkylene glycols or -thioglycols which have C2-C6-alkylene units, such as ethylene glycol, 1,2- or 1,3-propylene glycol, 1,2- or 1,4-butylene glycol, C1-C4-alkyl ethers of polyhydric alcohols, such as ethylene glycol monomethyl or monoethyl ethers, diethylene glycol monomethyl or monoethyl ethers, diethylene glycol monobutyl ether (butyl diglycol) or triethylene glycol monomethyl or monoethyl ethers, C1-C4-alkyl ethers of polyhydric alcohols, γ-butyrolactone or dimethyl sulfoxide or tetrahydrofuran. Preference is given to mixtures of the organic solvents with water, where the water content can be up to 95% by weight. Preference is given to a water content of 5-60% by weight.

The reaction of the diamines with the lactones is described in H. U. Geyer, Chem. Ber. 1964, 2271. In this connection, the molar ratio of aliphatic diamine to the polyhydroxy acid lactone can vary within a wide range, such as, for example, in the ratio 5:1 to 0.3:1, in particular 3:1 to 0.4:1. Preferably, the aliphatic diamine is added to the polyhydroxy acid lactone in a molar ratio of about 2:1 to 0.5:1.

The reaction according to the invention of the diamines with the lactones takes place in a temperature range from −5° C. to 50° C., preferably in a temperature range from 0° C. to 25° C. The reaction time is in the range from 2 to 30 hours, preferably in the range from 5 to 25 hours.

Any diamine which may be in excess during the reaction of the diamines with the lactones can be removed from the reaction mixture following the reaction in a suitable manner. Of suitability for this are preferably molecular sieves (pore size e.g. in the range from about 3-10 angstroms) or separating off by means of distillation or separating off by means of extraction with solvents or separating off with the help of suitable semipermeable membranes.

According to the invention, the molar ratio of anhydride to aminoalkylaldonamide can vary, e.g. in the ratio 1:0.8 to 1:1.2. The anhydride is preferably used in an approximately equimolar ratio relative to the aminoalkylaldonamide.

The reaction according to the invention of the aminoalkylaldonamide with the anhydride of a monounsaturated carboxylic acid takes place in the aforementioned organic solvents or solvent mixtures or the mixture of at least one organic solvent with water. Preferably, both reaction steps are carried out in one and the same solvent/solvent mixture or the mixture of the solvent with water, in particular without interim isolation of the reaction product.

The reaction according to the invention of the aminoalkylaldonamide with the anhydride of a monounsaturated carboxylic acid takes place in a temperature range from −5° C. to 50° C., preferably in a temperature range from 5° C. to 25° C. The reaction time is in the range from 2 to 10 hours, preferably in the range from 3 to 5 hours.

During the reaction procedure according to the invention, over and above the storage stabilizer which is present anyway in the anhydride compound, additional stabilizer may be added to the reaction mixture, for example hydroquinone monomethyl ether, phenothiazine, phenols, such as, for example, 2-tert-butyl-4-methyiphenol, 6-tert-butyl-2,4-dimethylphenol or N-oxyls, such as 4-hydroxy-2,2,6,6-tetramethylpiperidine-N-oxyl, 4-oxo-2,2,6,6-tetramethylpiperidine-N-oxyl or Uvinul® 4040P from BASF Aktiengesellschaft or amines such as Kerobit® BPD from BASF Aktiengesellschaft (N,N′-di-sec-butyl-p-phenylenediamine), for example in amounts of from 50 to 2000 ppm.

The reaction is advantageously carried out in the presence of an oxygen-containing gas, preferably air or air/nitrogen mixtures.

Preferably, the stabilizer (mixture) is used in the form of an aqueous solution.

The acid which may be produced during the amide formation from the acid anhydride, for example in the case of acrylic anhydride or methacrylic anhydride the acrylic acid or methacrylic acid, respectively, can be removed from the reaction mixture after the reaction in a suitable manner. Of suitability for this are preferably molecular sieves (pore size e.g. in the range from about 3-10 angstroms), or separating off by means of distillation or with the help of suitable semipermeable membranes. However, it is advantageous to co-use them directly as comonomer for the polymerization.

The process according to the invention is characterized by a simple and cost-effective reaction procedure. In this way, it is possible to avoid complex isolation processes prior to the further reaction. Instead, it is possible to use the resulting reaction mixture directly for the further polymerization.

The invention further provides novel unsaturated acylamidoalkylpolyhydroxy acid amides obtainable by reacting the reaction product of polyhydroxy acid lactone and aliphatic diamine with the anhydride of a monounsaturated carboxylic acid.

The novel unsaturated acylamidoalkylpolyhydroxy acid amides obey the general formula III

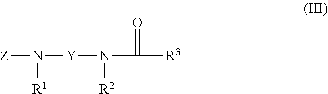

in which
Z is the radical of a saccharide oxidized on the anomeric carbon to the acid, the bonding of which takes place via the carbonyl function
R¹ and R² independently of one another are hydrogen or C₁-C₄-alkyl or C1-C4-hydroxyalkyl, in particular hydrogen or methyl
R³ is a vinyl radical which is optionally substituted by C₁-C₆-alkyl or carboxyl, or is an allyl radical which is optionally substituted by carboxyl, in particular vinyl or 2-propen-2-yl and
Y is C₂-C10-alkylene, which may optionally be interrupted by oxygen in the ether function and/or may be substituted by one or two carboxyl, hydroxyl and/or carboxamide groups, or is a cycloaliphatic radical.

Preferably, Z is a radical of the general formula IV

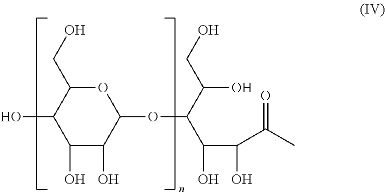

in which n is the number 0, 1, 2, 3, 4, 5, 6, 7 or 8.

In particular, Z is a radical derived from aldohexoses, preferably arabinose, ribose, xylose, mannose, galactose and in particular glucose.

In particular, Z is a radical derived from oligosaccharides such as lactose, maltose, isomaltose, maltotriose, maltotetraose and maltopentaose.

In particular, Z is a radical derived from a saccharide mixture obtainable through hydrolysis of a polysaccharide, such as hydrolysis of cellulose or starch.

The invention further provides a process for the preparation of polymers which comprise acylamidoalkylpolyhydroxy acid amide groups in copolymerized form, comprising the preparation of an unsaturated acylamidoalkylpolyhydroxy acid amide prepared according to the process of the invention, and the subsequent free-radical polymerization of the unsaturated acylamidoalkylpolyhydroxy acid amide, optionally together with monomers copolymerizable therewith. According to the process for the preparation of polymers comprising acylamidoalkylpolyhydroxy acid amide groups, at least one reaction product of polyhydroxy acid lactone and aliphatic diamine is reacted with the anhydride of a monounsaturated carboxylic acid, if necessary the unsaturated acylamidoalkylpolyhydroxy acid amide is separated off, and the reaction product is free-radically polymerized, optionally following the addition of comonomers. Preferably, for the polymerization, the reaction product of the reaction of aminoalkylaldonamide and anhydride of a monounsaturated carboxylic acid is used directly, if appropriate following the addition of monomers copolymerizable therewith.

Suitable further comonomers are: other unsaturated acylamidoalkylpolyhydroxy acid amides prepared according to the invention or polymerizable non-sugar monomers, such as (meth)acrylic acid, maleic acid, itaconic acid, alkali metal or ammonium salts thereof and esters thereof, O-vinyl esters of C₁-C₂₅-carboxylic acids, N-vinylamides of C₁-C₂₅-carboxylic acids, N-vinylpyrrolidone, N-vinylcaprolactam, N-vinyloxazolidone, N-vinylimidazole, (meth)acrylamide, (meth)acrylonitrile, ethylene, propylene, butylene, butadiene, styrene. Examples of suitable C1-C₂5-carboxylic acids are saturated acids, such as formic acid, acetic acid, propionic acid and n- and isobutyric acid, n- and isovaleric acid, caproic acid, oenanthoic acid, caprylic acid, pelargonic acid, capric acid, undecanoic acid, lauric acid, tridecanoic acid, myristic acid, pentadecanoic acid, palmitic acid, margaric acid, stearic acid, nonadecanoic acid, arachic acid, behenic acid, lignoceric acid, cerotic acid and melissic acid.

The preparation of such polymers takes place, for example, analogously to the processes described in general in “Ullmann's Enzyclopedia of Industrial Chemistry, Sixth Edition, 2000, Electronic Release, keyword: Polymerisation Process”. Preferably, the (co)polymerization takes place as a free-radical polymerization in the form of a solution polymerization, suspension polymerization, precipitation polymerization or emulsion polymerization or by bulk polymerization, i.e. without solvents.

The invention will now be illustrated in more detail by reference to the examples below.

### EXAMPLE 1

### Methacylamidoethylgluconamide

150.1 g (0.630 mol) of 1-amino-2-D-gluconoylaminoethane (prepared in accordance with: H. U. Geyer, Chem. Ber. 1964, 2271) and 1.45 g of hydroquinone monomethyl ether were dissolved in a mixture of 1080 g of methanol and 120 g of water. The mixture was cooled to 5° C. and 97.15 g (0.630 mol) of methacrylic anhydride were slowly added. When the addition was complete, the mixture was allowed to warm to a temperature of 20° C. over the course of 1 h and stirred for a further 2 hours at 20° C. This gave the product in the form of a colorless suspension.

The chemical constitution of the product was ascertained using 1 H-NMR and 13C-NMR spectroscopy. It was a mixture of methacylamidoethylgluconamide and methacrylic acid in the molar ratio 1:1.

### EXAMPLE 2

### N-Gluconylaminoethlymaleamide

400.0 g (1.68 mol) of 1-amino-2-D-gluconoylaminoethane (prepared in accordance with: H. U. Geyer, Chem. Ber. 1964, 2271) were dissolved in 404 g of water and adjusted to a pH of 6.5 by adding sulfuric acid. 164.7 g (1.68 mol) of maleic anhydride were dissolved in 384.4 g of acetone and then slowly added dropwise to the aqueous solution of 1-amino-2-D-gluconoylaminoethane. By adding sodium hydroxide solution, the pH was kept here at 6.5. When the addition was complete, the mixture was after-stirred for 2 hours at 20° C. Two liquid phases were formed. The lower phase was separated off. Acetone and water were distilled off in vacuo at 40-45° C. This gave 764 g of product in the form of a high-viscosity liquid. The chemical constitution of the product was ascertained using 1 H-NMR and 13C-NMR spectroscopy.

### Example 3

### N-Gluconyl-3-(N-methyl)aminopropylmethacrylamide

73.95 g (0.839 mol) of 3-methylaminopropylamine were dissolved in a mixture of 1440 g of methanol and 160 g of water. The mixture was cooled to 0° C. and, with stirring, at 0° C., 149.46 g (0.839 mol) of gluconolactone were slowly added. When the addition was complete, the mixture was stirred for 5 h at 0°-5° C. The mixture was then stirred for 17 h at 20° C. 1.45 g of a hydroquinone monomethyl ether were then added and the mixture was cooled to 5° C. Then, with stirring at 5° C., 129.40 g (0.90 mol) of methacrylic anhydride were slowly added. When the addition was complete, the mixture was allowed to warm to a temperature of 20° C. over the course of 1 h and stirred for a further 3 h at 20° C. The water and the methanol were distilled off in vacuo at 40° C. In the residue, the desired product was obtained, which still comprised a small amount of methacrylic acid.

The chemical constitution of the product was ascertained using 1 H-NMR and 13C-NMR spectroscopy.

1-12. (canceled)

13. A process for preparing an unsaturated acylamidoalkylpolyhydroxy acid amide, the process comprising:
reacting a reaction product of polyhydroxy acid lactone and aliphatic diamine with an anhydride of a monounsaturated carboxylic acid in a mixture of at least one organic solvent with water.

14. A process for preparing an unsaturated acylamidoalkylpolyhydroxy acid amide, the process comprising:
reacting a polyhydroxy acid lactone with an aliphatic diamine in aqueous medium, to obtain a reaction product; and
reacting the reaction product with an anhydride of a monounsaturated carboxylic acid.

15. The process of claim 13, wherein the polyhydroxy acid lactone is a lactone of a saccharide oxidized only at the anomeric center.

16. The process of claim 14, wherein the polyhydroxy acid lactone is a lactone of a saccharide oxidized only at the anomeric center.

17. The process of claim 13, wherein the polyhydroxy acid lactone is a lactone of gluconic acid.

18. The process of claim 14, wherein the polyhydroxy acid lactone is a lactone of gluconic acid.

19. The process of claim 15, wherein the saccharide is an oligosaccharide.

20. The process of claim 16, wherein the saccharide is an oligosaccharide.

21. The process of claim 13, wherein the polyhydroxy acid lactone is a compound of formula (II) 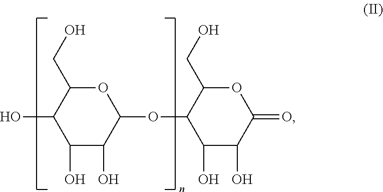
wherein n is 0, 1, 2, 3, 4, 5, 6, 7, or 8.

22. The process of claim 14, wherein the polyhydroxy acid lactone is a compound of formula (II) 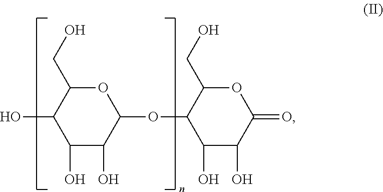
wherein n is 0, 1, 2, 3, 4, 5, 6, 7, or 8.

23. The process of claim 15, wherein the saccharide is obtained by a process comprising hydrolysis of a polysaccharide to obtain a hydrolysis product, and subsequent oxidation of the hydrolysis product.

24. The process of claim 16, wherein the saccharide is obtained by a process comprising hydrolysis of a polysaccharide to obtain a hydrolysis product, and subsequent oxidation of the hydrolysis product.

25. The process of claim 15, wherein the saccharide is obtained by a process comprising hydrolysis of cellulose or starch to obtain a hydrolysis product, and subsequent oxidation of the hydrolysis product.

26. The process of claim 16, wherein the saccharide is obtained by a process comprising hydrolysis of cellulose or starch to obtain a hydrolysis product, and subsequent oxidation of the hydrolysis product.

27. The process of claim 13, wherein the anhydride of the monounsaturated carboxylic acid is acrylic anhydride, methacrylic anhydride, itaconic anhydride, or maleic anhydride.

28. The process of claim 14, wherein the anhydride of the monounsaturated carboxylic acid is acrylic anhydride, methacrylic anhydride, itaconic anhydride, or maleic anhydride.

29. An unsaturated acylamidoalkylpolyhydroxy acid amide, obtained by a process comprising reacting a reaction product of polyhydroxy acid lactone of formula (II) 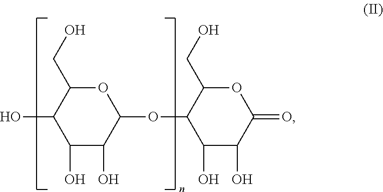
wherein n is 1, 2, 3, 4, 5, 6, 7, or 8,
and aliphatic diamine,
with the anhydride of a monounsaturated carboxylic acid.

30. An unsaturated acylamidoalkylpolyhydroxy acid amide of formula (III), 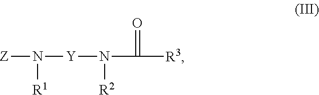
wherein
Z is a radical of a saccharide of formula (I) 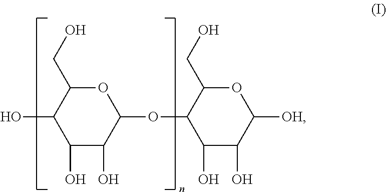
wherein n is 1, 2, 3, 4, 5, 6, 7, or 8,
which is oxidized on the anomeric carbon to the acid, bonding of which takes place via the carbonyl function,
R¹ and R² independently of one another are hydrogen, C₁-C₄-alkyl, or C₁-C₄-hydroxyalkyl,
R³ is a vinyl radical which is optionally substituted by C₁-C₆-alkyl or carboxyl or is an allyl radical which is optionally substituted by carboxyl, and
Y is C₂-C₁₀-alkylene, which is optionally interrupted by oxygen in an ether function and/or substituted by one or two carboxyl, hydroxyl and/or carboxamide groups, or is a cycloaliphatic radical.

31. A process for preparing a polymer comprising an acylamidoalkylpolyhydroxy acid amide group in copolymerized form, the process comprising:
free-radical polymerizing an unsaturated acylamidoalkylpolyhydroxy acid amide prepared by the process of claim 13, optionally together with at least one further monomer copolymerizable therewith.

32. A process for preparing a polymer comprising an acylamidoalkylpolyhydroxy acid amide group in copolymerized form, the process comprising:
free-radical polymerizing an unsaturated acylamidoalkylpolyhydroxy acid amide prepared by the process of claim 14, optionally together with at least one further monomer copolymerizable therewith.
